(19) United States
(12) Patent Application Publication
(10) Pub. No.: US 2014/0142150 A1
(43) Pub. Date: May 22, 2014

(54) TREATING NEUROPSYCHIATRIC DISORDERS

(71) Applicants: Donald C. Goff, Marblehead, MA (US); Joshua Roffman, Newton, MA (US)

(72) Inventors: Donald C. Goff, Marblehead, MA (US); Joshua Roffman, Newton, MA (US)

(73) Assignee: THE GENERAL HOSPITAL CORPORATION, Boston, MA (US)

(21) Appl. No.: 13/977,820

(22) PCT Filed: Jan. 23, 2012
(86) PCT No.: PCT/US2012/022240

§ 371 Date: Feb. 7, 2014

Related U.S. Application Data
(60) Provisional application No. 61/435,581, filed on Jan. 24, 2011.

(51) Int. Cl. C12Q 1/68

(52) U.S. Cl. CPC C12Q 1/6883
USPC 514/380; 435/6.11

(57) ABSTRACT

The specification provides methods of treating a subject suffering from a neuropsychiatric disorder and methods of determining whether a subject is suffering from or at risk for developing a neuropsychiatric disorder or likely to respond to a specified treatment method.

### CROSS-REFERENCE TO RELATED APPLICATION

This application claims priority under 35 U.S.C. §119(e) to U.S. Provisional Patent Application Ser. No. 61/435,581, filed on Jan. 24, 2011, the entire contents of which are hereby incorporated by reference in its entirety.

### FEDERALLY SPONSORED RESEARCH OR DEVELOPMENT

This invention was made with Government support under Grant Number K24 MH002025 awarded by National Institute of Mental Health. The Government has certain rights in the invention.

### TECHNICAL FIELD

The claimed methods relate to genetic markers of neuropsychiatric disorders and methods of use thereof.

### BACKGROUND

N-Methyl-D-aspartate (NMDA) receptors play critical roles in the development, function, and death of neurons. These ionotropic receptors allow for electrical signals to transfer between neurons in the brain and in the spinal column. To conduct an electrical signal, the NMDA receptor must be activated by glutamate or aspartate. In addition, NMDA receptors require binding of an agonist at the glycine binding site for efficient opening of the ion channel. Glycine, D-alanine, and D-serine are endogenous agonists at the glycine site; the antimicrobial agent, D-cycloserine, is a partial agonist at the NMDA receptor. Activation of the glycine site can also be enhanced by blocking glycine reuptake with glycine transporter inhibitors. Function of the NMDA receptor is compromised in many neuropsychiatric disorders, including schizophrenia, Alzheimer's Disease, autism, depression, and attention deficit disorder. The term schizophrenia represents a group of neuropsychiatric disorders characterized by dysfunctions of the thinking process, such as delusions, hallucinations, and extensive withdrawal of the subject's interests from other people. Approximately one percent of the worldwide population is afflicted with schizophrenia, and this disorder is accompanied by high morbidity and mortality rates. Alzheimer's Disease is a form of dementia that typically involves progressive mental deterioration, manifested by memory loss, confusion, and disorientation. Alzheimer's Disease typically is treated by acetylcholine esterase inhibitors such as tacrine hydrochloride or donepezil. Autism is a developmental mental disorder characterized by autistic behavior, social failure, and language delay. Depression is a clinical syndrome that includes a persistent sad mood or loss of interest in activities, which persists for at least two weeks in the absence of treatment. Conventional therapeutics include serotonin uptake inhibitors (e.g., PROZAC® (fluoxetine)), monoamine oxidase inhibitors, and tricyclic antidepressants. Attention Deficit Disorder is a disorder that is most prevalent in children and is associated with increased motor activity and a decreased attention span. Attention Deficit Disorder commonly is treated by administration of psychostimulants such as RITALIN® (methylphenidate) or dexedrine. Although numerous options for pharmacologic treatment for neuropsychiatric disorders are available, current treatments continue to have limitations of both efficacy and tolerability. Therefore, effective methods of selecting an appropriate treatment for a subject suffering from a neuropsychiatric disorder are desirable.

### SUMMARY

Methods to predict response to treatments that act directly or indirectly at the glycine site of an NMDA receptor and other treatments for neuropsychiatric disorders, e.g., schizophrenia, Alzheimer's Disease, autism, depression, and attention deficit disorder, are described. The present specification provides a panel of single nucleotide polymorphism (SNP) biomarkers for predicting the response to treatment. In one aspect, the methods described herein feature methods of selecting an appropriate treatment for a subject based on a presence of one or more alleles at rs3916971 and rs202676 in genomic DNA.

In one aspect, the methods described herein feature methods of treating a subject, e.g., a human, diagnosed as having a neuropsychiatric disorder characterized by attenuated NMDA neurotransmission are provided. The methods include determining the presence of one or more alleles at rs3916971 and rs202676 in a sample comprising genomic DNA from the subject, e.g., plasma or whole blood, selecting a treatment for the subject based on the presence of the one or more alleles, and treating the subject with the selected treatment.

In one embodiment, the neuropsychiatric disorder is schizophrenia. In some embodiments, the subject is diagnosed as having a negative symptom of schizophrenia, e.g., apathy, impoverished speech, flattened affect, social withdrawal, or any combination thereof. In some embodiments, the neuropsychiatric disorder is selected from the group consisting of Alzheimer's disease, autism, depression, and attention deficit disorder.

In one embodiment, if a “T” at rs3916971 or a “C” at rs202676 is present, then a treatment comprising prescribing or administering an agonist of the glycine site of an NMDA receptor to the subject is selected.

In some embodiments, if a “T” at rs3916971 and a “C” at rs202676 are present, then a treatment comprising prescribing or administering an agonist of the glycine site of an NMDA receptor to the subject is selected.

In some embodiments, the method comprises detecting the presence of four alleles, wherein the four alleles consist of two alleles at each of rs3916971 and rs202676. In one embodiment, if a “T” at rs3916971 and a “C” at rs202676 are present, and one or more additional alleles are a “T” at rs3916971 or a “C” at rs202676, then a treatment comprising prescribing or administering an agonist of the glycine site of an NMDA receptor to the subject is selected. In one embodiment, if two alleles are a “T” at rs3916971 and two alleles are a “C” at rs202676, then a treatment comprising prescribing or administering an agonist of the glycine site of an NMDA receptor to the subject is selected.

In some embodiments, the selected treatment includes prescribing or administering an agonist of the glycine site of an NMDA receptor, e.g., one or more of glycine, a salt of glycine, an ester of glycine, alkylated glycine, a precursor of glycine, D-alanine, a salt of D-alanine, an ester of D-alanine, alkylated D-alanine, a precursor of D-alanine, D-serine, a salt of D-serine, an ester of D-serine, alkylated D-serine, and/or a precursor of D-serine, to the subject.

In one embodiment, the selected treatment includes prescribing or administering an NMDA receptor partial agonist, e.g., one or more of D-cycloserine, a salt of D-cycloserine, an ester of D-cycloserine, alkylated D-cycloserine, and/or a precursor of D-cycloserine, to the subject.

In one embodiment, the selected treatment includes prescribing or administering a glycine reuptake inhibitor, e.g., one or more of N-methylglycine, a salt of N-methylglycine, an ester of N-methylglycine, alkylated N-methylglycine, a precursor of N-methylglycine, and/or RG1678, to the subject.

In one embodiment, the selected treatment further comprises prescribing or administering an antipsychotic, e.g., one or more of haloperidol, chlorpromazine, triflupromazine, chlorprothixene, thiothixene, clozapine, risperidone, and/or aripiprazole, to the subject.

In one aspect, the methods described herein include assaying for the presence of one or more alleles at rs3916971 and rs202676 in a biological sample comprising genomic DNA from a subject diagnosed as having a neuropsychiatric disorder characterized by attenuated NMDA neurotransmission and transmitting to a recipient, e.g., health care provider, medical caregiver, physician, and nurse, a report on the presence of the one or more alleles.

In some embodiments, the biological sample comprising genomic DNA can be, e.g., plasma or whole blood, from the subject, e.g., a human.

In one embodiment, the neuropsychiatric disorder is schizophrenia. In some embodiments, the subject is diagnosed as having a negative symptom of schizophrenia, e.g., apathy, impoverished speech, flattened affect, social withdrawal, or any combination thereof. In some embodiments, the neuropsychiatric disorder is selected from the group consisting of Alzheimer's disease, autism, depression, and attention deficit disorder.

In one embodiment, the methods include selecting a treatment for reducing the negative symptom in the subject based on the presence of the one or more alleles. In some embodiments, the selected treatment includes prescribing or administering an agonist of the glycine site of an NMDA receptor to the subject.

In some embodiments, the selected treatment includes prescribing or administering an agonist of the glycine site of an NMDA receptor, e.g., one or more of glycine, a salt of glycine, an ester of glycine, alkylated glycine, a precursor of glycine, D-alanine, a salt of D-alanine, an ester of D-alanine, alkylated D-alanine, a precursor of D-alanine, D-serine, a salt of D-serine, an ester of D-serine, alkylated D-serine, and/or a precursor of D-serine, to the subject.

In one embodiment, the selected treatment includes prescribing or administering an NMDA receptor partial agonist, e.g., one or more of D-cycloserine, a salt of D-cycloserine, an ester of D-cycloserine, alkylated D-cycloserine, and/or a precursor of D-cycloserine, to the subject.

In one embodiment, the selected treatment includes prescribing or administering a glycine reuptake inhibitor, e.g., one or more of N-methylglycine, a salt of N-methylglycine, an ester of N-methylglycine, alkylated N-methylglycine, a precursor of N-methylglycine, and/or RG1678, to the subject.

In one embodiment, if a “T” at rs3916971 or a “C” at rs202676 is present, then a treatment comprising prescribing or administering an agonist of the glycine site of an NMDA receptor to the subject is selected.

In some embodiments, if a “T” at rs3916971 and a “C” at rs202676 are present, then a treatment comprising prescribing or administering an agonist of the glycine site of an NMDA receptor to the subject is selected.

In some embodiments, the method comprises detecting the presence of four alleles, wherein the four alleles consist of two alleles at each of rs3916971 and rs202676. In one embodiment, if a “T” at rs3916971 and a “C” at rs202676 are present, and one or more additional alleles are a “T” at rs3916971 or a “C” at rs202676, then a treatment comprising prescribing or administering an agonist of the glycine site of an NMDA receptor to the subject is selected. In one embodiment, if two alleles are a “T” at rs3916971 and two alleles are a “C” at rs202676, then a treatment comprising prescribing or administering an agonist of the glycine site of an NMDA receptor to the subject is selected.

In yet another aspect, a plurality of polynucleotides bound to a solid support are provided. Each polynucleotide of the plurality selectively hybridizes to one or more SNP alleles selected from the group consisting of rs3916971 and rs202676.

In some embodiments, the plurality of polynucleotides comprise SEQ ID NOs:3, 4, 5, 6, 7, and/or 8, or any combination thereof

In some aspects, the specification provides nucleotide sequences, e.g., polynucleotides comprising the sequences of SEQ ID NOs:3, 4, 5, 6, 7, and/or 8, or any combination thereof, to detect a presence of one or more alleles at rs3916971 and rs202676.

As used herein, the term “neuropsychiatric disorder” refers to a condition having a pathophysiological component of attenuated NMDA receptor-mediated neurotransmission. Examples of such disorders include schizophrenia, Alzheimer's disease, autism, depression, and attention deficit disorder. These exemplary neuropsychiatric disorders and their symptoms are well-known in the art and are described in further detail below.

As used herein, the term “schizophrenia” refers to a psychiatric disorder that includes at least one of the following: delusions, hallucinations, disorganized speech, grossly disorganized or catatonic behavior, or negative symptoms (e.g., apathy, impoverished speech, flattened affect, and social withdrawal). Patients can be diagnosed as schizophrenic using the DSM-IV criteria (American Psychiatric Association, 1994, Diagnostic and Statistical Manual of Mental Disorders (Fourth Edition), Washington, D.C.). Patients can be diagnosed as having a negative symptom of schizophrenia by a health care provider, medical caregiver, physician, nurse, family member, or acquaintance, who recognizes, appreciates, acknowledges, determines, concludes, opines, or decides that the subject has a negative symptom of schizophrenia.

If desired, one can measure negative and/or positive and/or cognitive symptom(s) of schizophrenia before and after treatment of the subject. A reduction in such a symptom indicates that the subject's condition has improved. Improvement in the symptoms of schizophrenia can be assessed using the Scales for the Assessment of Negative Symptoms (SANS), Iowa City, Iowa and Kay et al., 1987, Schizophrenia Bulletin 13:261-276) or Positive and Negative Syndrome Scale (PANSS) (see, e.g., Andreasen, 1983).

The term “Alzheimer's Disease” refers to a progressive mental deterioration manifested by memory loss, confusion and disorientation beginning in late middle life and typically resulting in death in five to ten years. Pathologically, Alzheimer's Disease can be characterized by thickening, conglutination, and distortion of the intracellular neurofibrils, neurofibrillary tangles and senile plaques composed of granular or filamentous argentophilic masses with an amyloid core. Methods for diagnosing Alzheimer's Disease are known in the art. For example, the National Institute of Neurological and Communicative Disorders and Stroke-Alzheimer's Disease and the Alzheimer's Disease and Related Disorders Association (NINCDS-ADRDA) criteria can be used to diagnose Alzheimer's Disease (McKhann et al., Neurology 34:939-944, 1984). The subject's cognitive function can be assessed by the Alzheimer's Disease Assessment Scale-cognitive subscale (ADAS-cog; Rosen et al., Am. J. Psychiatry 141:1356-1364, 1984).

As used herein, the term “autism” refers to a state of mental introversion characterized by morbid self-absorption, social failure, language delay, and stereotyped behavior. Subjects can be diagnosed as suffering from autism by using the DSM-IV criteria.

As used herein, the term “depression” refers to a clinical syndrome that includes a persistent sad mood or loss of interest in activities, which lasts for at least one week in the absence of treatment. The DSM-IV criteria can be used to diagnose subjects as suffering from depression.

The term “attention deficit disorder,” as used herein, refers to a disorder that is most commonly exhibited by children and which can be characterized by increased motor activity and a decreased attention span. The DSM-IV criteria can be used to diagnose attention deficit disorder.

The terms “D-alanine” and “D-serine” refer to the D isomers of the amino acids alanine and serine, respectively. D-isomers, as opposed to L-isomers, are not naturally found in proteins.

As used herein, an “allele” is one of a pair or series of genetic variants of a polymorphism at a specific genomic location. A “schizophrenia susceptibility allele” is an allele that is associated with increased susceptibility of developing schizophrenia.

As used herein, a “haplotype” is one or a set of signature genetic changes (polymorphisms) that are normally grouped closely together on the DNA strand, and are usually inherited as a group; the polymorphisms are also referred to herein as “markers.” A haplotype is information regarding the presence or absence of one or more genetic markers in a given chromosomal region in a subject. A haplotype can consist of a variety of genetic markers, including indels (insertions or deletions of the DNA at particular locations on the chromosome); SNPs in which a particular nucleotide is changed; microsatellites; and minisatellites.

As used herein, an “based on” refers to taking the presence of one or more alleles, e.g., at rs3916971 and rs202676, into consideration or accounting for the presence of one or more alleles, e.g., at rs3916971 and rs202676.

“Linkage disequilibrium” refers to when the observed frequencies of haplotypes in a population does not agree with haplotype frequencies predicted by multiplying together the frequency of individual genetic markers in each haplotype.

The term “chromosome” as used herein refers to a gene carrier of a cell that is derived from chromatin and comprises DNA and protein components (e.g., histones). The conventional internationally recognized individual human genome chromosome numbering identification system is employed herein. The size of an individual chromosome can vary from one type to another with a given multi-chromosomal genome and from one genome to another. In the case of the human genome, the entire DNA mass of a given chromosome is usually greater than about 100,000,000 base pairs. For example, the size of the entire human genome is about 3×10⁹ base pairs.

The term “gene” refers to a DNA sequence in a chromosome that codes for a product (either RNA or its translation product, a polypeptide). A gene contains a coding region and includes regions preceding and following the coding region (termed respectively “leader” and “trailer”). The coding region is comprised of a plurality of coding segments (“exons”) and intervening sequences (“introns”) between individual coding segments.

The term “probe” refers to an oligonucleotide. A probe can be single stranded at the time of hybridization to a target. As used herein, probes include primers, i.e., oligonucleotides that can be used to prime a reaction, e.g., a PCR reaction.

Unless otherwise defined, all technical terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a bar graph showing the mean SANS change from D-cycloserine treatment based on G72 (rs3916971) genotype.

FIG. 2 is a bar graph depicting the mean SANS change from D-cycloserine treatment based on GCPII (rs202676) genotype.

FIG. 3 is a scatter plot showing the relationship between SANS change and risk allele load.

### DETAILED DESCRIPTION

NMDA receptor hypofunction has been identified as a mechanism underlying psychosis, negative symptoms, and cognitive deficits in schizophrenia, based in part on the production by NMDA antagonists of all three symptom domains in healthy subjects. Agonists at the glycine site of the NMDA receptor have improved negative symptoms in some studies, but the failure to produce consistent results and the lack of efficacy for cognition and psychosis has been puzzling. Recent findings suggest that daily dosing with glycine site agonists produces endocytosis of NMDA receptors and rapid loss of clinical efficacy, whereas intermittent dosing promotes neuroplasticity—the persistent enhancement of synaptic efficiency. It is well established that a single dose of the glycine site partial agonist, D-cycloserine, enhances learning in animal models, but tolerance develops with repeated daily dosing. Similarly, once-weekly dosing of D-cycloserine produces persistent improvement when combined with cognitive behavioral therapy (CBT) in anxiety disorders.

The methods described herein are based, at least in part, on markers that are associated with neuropsychiatric disorders characterized by attenuated NMDA neurotransmission, e.g., a negative symptoms of schizophrenia (Table 1). Methods to predict response to agents acting directly or indirectly (e.g., glycine uptake inhibitors) at the glycine site of the NMDA receptor are presented. Analysis provided evidence of an association of the disclosed SNPs and negative symptoms of schizophrenia. A SNP occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations). A SNP usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele. Typically the polymorphic site is occupied by a base other than the reference base. For example, where the reference allele contains the base “C” at the polymorphic site, the altered allele can contain a “T,” “G,” or “A” at the polymorphic site.

TABLE 1
SNPs Associated with Negative
Symptoms of Schizophrenia

| | | | Risk |
|---|---|---|---|
| SNP | Location | Sequence | Allele |
| G72 | 13q34 | ACACCTGGCACATAGTAAT | T |
| rs3916971 | | AGATCAT[C/T]AAAT | |
| | | GTGAGCAAGGATTAGTTGCCA | |
| | | (SEQ ID NO: 1) | |
| GCPII | 11p11 | AAGCTGAGAACATCAAGAAG | C |
| rs202676 | | TTCTTA[C/T]AGTAAGTA | |
| | | CATCCTCGAAAGTTTAT | |
| | | (SEQ ID NO: 2) | |

A series of SNP risk alleles have been identified that are associated with negative symptoms of schizophrenia. The presence of one or more of these SNP risk alleles, e.g., two, three, or four alleles described in Table 1, can be used to determine whether a subject is suffering from or at risk for developing a negative symptom of schizophrenia. The presence of one or more SNP risk alleles, e.g., two, three, or four alleles described in Table 1, can be used select a treatment, e.g., an agonist of the glycine site of an NMDA receptor, for a subject suffering from a negative symptom of schizophrenia. The SNP genotypes (identified by their SNP site and alleles) are depicted in Table 1. Further information on the SNPs can be obtained from, for example, the National Center for Biotechnology Information Entrez Single Nucleotide Polymorphism database that is accessible via the Internet. Genetic variation in genes associated with NMDA receptor function contributes to negative symptoms in schizophrenia. Missense variants in two genes, G72 and GCPII, are independently associated with negative symptom scores. Moreover, the specification provides evidence of a cumulative effect of risk variants in G72 and GCPII, where patients who carry more than one, e.g., two, three, or four risk alleles across the two genes exhibited a stronger inverse relationship with negative symptom scores.

### Methods for Determining Susceptibility to a Neuropsychiatric Disorder

Described herein are a variety of methods of treating a subject suffering from a neuropsychiatric disorder, e.g., a negative symptom of schizophrenia, and methods of determining whether a subject is suffering from or at risk for developing a neuropsychiatric disorder, e.g., a negative symptom of schizophrenia. An increased susceptibility to a neuropsychiatric disorder, e.g., a negative symptom of schizophrenia, exists if a subject has an allele or a haplotype associated with an increased susceptibility to a neuropsychiatric disorder, e.g., a negative symptom of schizophrenia, i.e., a “risk allele,” as described in Table 1. Ascertaining or assaying whether the subject has such a risk allele or a haplotype is included in the concept of determining susceptibility to a neuropsychiatric disorder, e.g., a negative symptom of schizophrenia. Such determination is useful, for example, for purposes of diagnosis, treatment selection (e.g., of the same, new, or different treatments), and genetic counseling. Thus, the methods described herein can include detecting an allele or a haplotype associated with an increased susceptibility to a neuropsychiatric disorder, e.g., a negative symptom of schizophrenia, as described herein for the subject.

### Methods of Treating a Subject Having a Neuropsychiatric Disorder

Described herein are a variety of methods of treating a subject having a neuropsychiatric disorder characterized by attenuated NMDA neurotransmission, e.g., a subject diagnosed as having a negative symptom of schizophrenia. A decrease in negative symptoms of schizophrenia in response to treatment with an agonist of the glycine site of an NMDA receptor results if a subject has an allele or a haplotype associated with a “risk allele,” as described in Table 1. Ascertaining or assaying whether the subject has such a risk allele or a haplotype is included in the concept of treating a subject having a neuropsychiatric disorder characterized by attenuated NMDA neurotransmission. Such determination is useful, for example, for purposes of diagnosis, treatment selection (e.g., an agonist of the glycine site of an NMDA receptor, and new or different treatments), and genetic counseling. Thus, the methods described herein can include assaying or detecting an allele or a haplotype associated with a decrease in negative symptoms of schizophrenia in response to treatment with an agonist of the glycine site of an NMDA receptor as described herein for the subject.

Methods of Determining the Presence or Absence of an Allele or a Haplotype Associated with a Neuropsychiatric Disorder

The methods described herein include determining the presence or absence of alleles or haplotypes associated with a neuropsychiatric disorder, e.g., a negative symptom of schizophrenia. In some embodiments, an association with a negative symptom of schizophrenia is determined by the presence of a shared haplotype between the subject and an affected reference individual, e.g., a first or second-degree relation of the subject, and the absence of the haplotype in an unaffected reference individual. Thus the methods can include obtaining and analyzing a sample from a suitable reference individual.

Samples that are suitable for use in the methods described herein contain genetic material, e.g., genomic DNA (gDNA). Non-limiting examples of sources of samples include urine, blood, plasma, serum, saliva, semen, sputum, cerebral spinal fluid, tears, or mucus, or such a sample absorbed onto a paper or polymer substrate. A biological sample can be further fractionated, if desired, to a fraction containing particular cell types. For example, a blood sample can be fractionated into serum or into fractions containing particular types of blood cells such as red blood cells or white blood cells (leukocytes). If desired, a sample can be a combination of samples from a subject such as a combination of a tissue and fluid sample. The sample itself will typically consist of nucleated cells (e.g., blood or buccal cells), tissue, etc., removed from the subject. The subject can be an adult, child, fetus, or embryo. In some embodiments, the sample is obtained prenatally, either from a fetus or embryo or from the mother (e.g., from fetal or embryonic cells in the maternal circulation). Methods and reagents are known in the art for obtaining, processing, and analyzing samples. In some embodiments, the sample is obtained with the assistance of a health care provider, e.g., to draw blood. In some embodiments, the sample is obtained without the assistance of a health care provider, e.g., where the sample is obtained non-invasively, such as a sample comprising buccal cells that is obtained using a buccal swab or brush, or a saliva sample.

The sample may be processed before the detecting step. For example, DNA in a cell or tissue sample can be separated from other components of the sample. The sample can be concentrated and/or purified to isolate DNA. Cells can be harvested from a biological sample using standard techniques known in the art. For example, cells can be harvested by centrifuging a cell sample and resuspending the pelleted cells. The cells can be resuspended in a buffered solution such as phosphate-buffered saline (PBS). After centrifuging the cell suspension to obtain a cell pellet, the cells can be lysed to extract DNA, e.g., gDNA. See, e.g., Ausubel et al., 2003, supra. All samples obtained from a subject, including those subjected to any sort of further processing, are considered to be obtained from the subject.

The absence or presence of a haplotype associated with schizophrenia as described herein can be determined using methods known in the art, e.g., gel electrophoresis, capillary electrophoresis, size exclusion chromatography, sequencing, and/or arrays to detect the presence or absence of the marker(s) of the haplotype. Amplification of nucleic acids, where desirable, can be accomplished using methods known in the art, e.g., PCR.

As used herein, “detecting an allele or a haplotype,” “determining the presence of one or more alleles,” and “assaying for the presence of one or more alleles” includes obtaining information regarding the identity, presence or absence of one or more genetic markers in a subject. Detecting an allele or a haplotype, determining or assaying for the presence of one or more alleles can, but need not, include obtaining a sample comprising DNA from a subject, and/or assessing the identity, presence or absence of one or more genetic markers in the sample. The individual or organization who detects, determines, or assays the allele or haplotype need not actually carry out the physical analysis of a sample from a subject; the information can be obtained by analysis of the sample by a third party. Thus the methods can include steps that occur at more than one site. For example, a sample can be obtained from a subject at a first site, such as at a health care provider, or at the subject's home in the case of a self-testing kit. The sample can be analyzed at the same or a second site, e.g., at a laboratory or other testing facility.

Detecting an allele or a haplotype and determining the presence of one or more alleles can also include or consist of reviewing a subject's medical history, where the medical history includes information regarding the identity, presence or absence of one or more genetic markers in the subject, e.g., results of a genetic test.

In some embodiments, to determine the presence of an allele or a haplotype described herein, a biological sample that includes nucleated cells (such as blood, a cheek swab, or saliva) is prepared and analyzed for the presence or absence of preselected markers. Such diagnoses may be performed by diagnostic laboratories. Alternatively, diagnostic kits containing probes or nucleic acid arrays useful in, e.g., determining the presence of one or more SNP alleles can be manufactured and sold to health care providers or to private individuals for self-diagnosis. Diagnostic or prognostic tests can be performed as described herein or using well known techniques, such as described in U.S. Pat. No. 5,800,998.

Results of these tests, and optionally interpretive information, can be returned to the subject, the health care provider, medical caregiver, physician, nurse, or to a third party payor. The results can be used in a number of ways. The information can be, e.g., communicated to the tested subject, e.g., with a prognosis and optionally interpretive materials that help the subject understand the test results and prognosis. The information can be used, e.g., by a health care provider, to determine whether to administer a specific drug, or whether a subject should be assigned to a specific category, e.g., a category associated with a specific disease phenotype, or with drug response or non-response. The information can be used, e.g., by a third party payor such as a healthcare payor (e.g., insurance company or HMO) or other agency, to determine whether or not to reimburse a health care provider for services to the subject, or whether to approve the provision of services to the subject. For example, the healthcare payor may decide to reimburse a health care provider for treatments for a neuropsychiatric disorder, e.g., schizophrenia, if the subject has an increased severity of negative symptoms of schizophrenia, e.g., a subject with one, two, three, or four risk alleles described in Table 1. As another example, a drug or treatment may be indicated for individuals with a certain haplotype, and the insurance company would only reimburse the health care provider (or the insured individual) for prescription or purchase of the drug if the insured individual has that haplotype. The presence or absence of the haplotype in a subject may be ascertained by using any of the methods described herein.

Information gleaned from the methods described herein can also be used to select or stratify subjects for a clinical trial. For example, the presence of a selected haplotype described herein can be used to select a subject for a trial. The information can optionally be correlated with clinical information about the patient, e.g., diagnostic or prognostic information.

### Linkage Disequilibrium Analysis

One of skill in the art will appreciate that markers within one Linkage Disequilibrium Unit (LDU) of the polymorphisms described herein can also be used in a similar manner to those described herein. Linkage disequilibrium (LD) is a measure of the degree of association between alleles in a population. LDUs share an inverse relationship with LD so that regions with high LD (such as haplotype blocks) have few

LDUs and low recombination, while regions with many LDUs have low LD and high recombination. Methods of calculating LDUs are known in the art (see, e.g., Morton et al., Proc Natl Acad Sci USA 98(9):5217-21 (2001); Tapper et al., Proc Natl Acad Sci USA 102(33):11835-11839 (2005); Maniatis et al., Proc Natl Acad Sci USA 99:2228-2233 (2002)). Thus, in some embodiments, the methods include analysis of polymorphisms that are within one LDU of a polymorphism described herein.

Alternatively, methods described herein can include analysis of polymorphisms that are within a value defined by Lewontin's D′ (linkage disequilibrium parameter, see Lewontin, Genetics 49:49-67 (1964)) of a polymorphism described herein. Results can be obtained, e.g., from on line public resources such as HapMap.org. The simple linkage disequilibrium parameter (D) reflects the degree to which alleles at two loci (for example two SNPs) occur together more often (positive values) or less often (negative values) than expected in a population as determined by the products of their respective allele frequencies. For any two loci, D can vary in value from -0.25 to +0.25. However, the magnitude of D (Dmax) varies as function of allele frequencies. To control for this, Lewontin introduced the D′ parameter, which is D/Dmax and varies in value from -1 (alleles never observed together) to +1 (alleles always observed together). Typically, the absolute value of D′ (i.e., |D′|) is reported in online databases, because it follows mathematically that positive association for one set of alleles at two loci corresponds to a negative association of equal magnitude for the reciprocal set. This disequilibrium parameter varies from 0 (no association of alleles at the two loci) to 1 (maximal possible association of alleles at the two loci).

Thus, in some embodiments, the methods include analysis of polymorphisms that are in complete linkage disequilibrium, i.e., with an R²=1 or a D′=1, for pairwise comparisons, of a polymorphism described herein.

Methods are known in the art for identifying suitable polymorphisms; for example, the International HapMap Project provides a public database that can be used, see, hapmap.org, as well as The International HapMap Consortium, Nature 426:789-796 (2003), and The International HapMap Consortium, Nature 437:1299-1320 (2005). Generally, it will be desirable to use a HapMap constructed using data from individuals who share ethnicity with the subject, e.g., a HapMap for Caucasians would ideally be used to identify markers within one LDU or with an R²=1 or D′=1 of a marker described herein for use in genotyping a subject of Caucasian descent.

### Identification of Additional Markers for Use in the Methods Described Herein

Skilled practitioners will also appreciate that additional markers can be used. In general, genetic markers can be identified using any of a number of methods well known in the art. For example, numerous polymorphisms in the regions described herein are known to exist and are available in public databases, which can be searched using methods and algorithms known in the art. Alternately, polymorphisms can be identified by sequencing either genomic DNA or cDNA in the region in which it is desired to find a polymorphism. According to one approach, primers are designed to amplify such a region, and DNA from a subject is obtained and amplified. The DNA is sequenced, and the sequence (referred to as a “subject sequence” or “test sequence”) is compared with a reference sequence, which can represent the “normal” or “wild type” sequence, or the “affected” sequence. In some embodiments, a reference sequence can be from, for example, the human draft genome sequence, publicly available in various databases, or a sequence deposited in a database such as GenBank. In some embodiments, the reference sequence is a composite of ethnically diverse individuals.

In general, if sequencing reveals a difference between the sequenced region and the reference sequence, a polymorphism has been identified. The fact that a difference in nucleotide sequence is identified at a particular site determines that a polymorphism exists at that site. In most instances, particularly in the case of SNPs, only two polymorphic variants will exist at any location. However, in the case of SNPs, up to four variants may exist since there are four naturally occurring nucleotides in DNA. Other polymorphisms, such as insertions and deletions, may have more than four alleles.

Methods of nucleic acid analysis to assay for polymorphisms and/or polymorphic variants include, e.g., microarray analysis. Hybridization methods, such as Southern analysis, Northern analysis, or in situ hybridizations, can also be used (see Current Protocols in Molecular Biology, Ausubel et al., Eds., John Wiley & Sons, 2003). To assay for microdeletions, fluorescence in situ hybridization (FISH) using DNA probes that are directed to a putatively deleted region in a chromosome can be used. For example, probes that detect all or a part of a microsatellite marker can be used to detect microdeletions in the region that contains that marker.

Other methods include direct manual sequencing (Church and Gilbert, Proc. Natl. Acad. Sci. USA 81:1991-1995 (1988); Sanger et al., Proc. Natl. Acad. Sci. 74:5463-5467 (1977); Beavis et al., U.S. Pat. No. 5,288,644); automated fluorescent sequencing; single-stranded conformation polymorphism assays (SSCP); clamped denaturing gel electrophoresis (CDGE); two-dimensional gel electrophoresis (2DGE or TDGE); conformational sensitive gel electrophoresis (CSGE); denaturing gradient gel electrophoresis (DGGE) (Sheffield et al., Proc. Natl. Acad. Sci. USA 86:232-236 (1989)), mobility shift analysis (Orita et al., Proc. Natl. Acad. Sci. USA 86:2766-2770 (1989)), restriction enzyme analysis (Flavell et al., Cell 15:25 (1978); Geever et al., Proc. Natl. Acad. Sci. USA 78:5081 (1981)); quantitative real-time PCR (Raca et al., Genet Test 8(4):387-94 (2004)); heteroduplex analysis; chemical mismatch cleavage (CMC) (Cotton et al., Proc. Natl. Acad. Sci. USA 85:4397-4401 (1985)); RNase protection assays (Myers et al., Science 230:1242 (1985)); use of polypeptides that recognize nucleotide mismatches, e.g., E. coli mutS protein; allele-specific PCR, for example. See, e.g., Gerber et al., U.S. Patent Publication No. 2004/0014095, which is incorporated herein by reference in its entirety. In some embodiments, the sequence is determined on both strands of DNA.

In order to assay for polymorphisms and/or polymorphic variants, it will frequently be desirable to amplify a portion of genomic DNA (gDNA) encompassing the polymorphic site. Such regions can be amplified and isolated by PCR using oligonucleotide primers designed based on genomic and/or cDNA sequences that flank the site. See, e.g., PCR Primer: A Laboratory Manual, Dieffenbach and Dveksler, (Eds.); McPherson et al., PCR Basics: From Background to Bench (Springer Verlag, 2000); Mattila et al., Nucleic Acids Res., 19:4967 (1991); Eckert et al., PCR Methods and Applications, 1:17 (1991); PCR (Eds. McPherson et al., IRL Press, Oxford); and U.S. Pat. No. 4,683,202. Other amplification methods that may be employed include the ligase chain reaction (LCR) (Wu and Wallace, Genomics, 4:560 (1989), Landegren et al., Science, 241:1077 (1988), transcription amplification (Kwoh et al., Proc. Natl. Acad. Sci. USA, 86:1173 (1989)), self-sustained sequence replication (Guatelli et al., Proc. Nat. Acad. Sci. USA, 87:1874 (1990)), and nucleic acid based sequence amplification (NASBA). Guidelines for selecting primers for PCR amplification are well known in the art. See, e.g., McPherson et al., PCR Basics: From Background to Bench, Springer-Verlag, 2000. A variety of computer programs for designing primers are available, e.g., ‘Oligo’ (National Biosciences, Inc, Plymouth Minn.), MacVector (Kodak/IBI), and the GCG suite of sequence analysis programs (Genetics Computer Group, Madison, Wis.).

In one example, a sample (e.g., a sample comprising genomic DNA), is obtained from a subject. The DNA in the sample is then examined to assay for an allele or a haplotype as described herein. The allele or haplotype can be detected by any method described herein, e.g., by sequencing or by hybridization of the gene in the genomic DNA, RNA, or cDNA to a nucleic acid probe, e.g., a DNA probe (which includes cDNA and oligonucleotide probes) or an RNA probe. The nucleic acid probe can be designed to specifically or preferentially hybridize with a particular polymorphic variant.

In some embodiments, a peptide nucleic acid (PNA) probe can be used instead of a nucleic acid probe in the hybridization methods described above. PNA is a DNA mimetic with a peptide-like, inorganic backbone, e.g., N-(2-aminoethyl)glycine units, with an organic base (A, G, C, T, or U) attached to the glycine nitrogen via a methylene carbonyl linker (see, e.g., Nielsen et al., Bioconjugate Chemistry, The American Chemical Society, 5:1 (1994)). The PNA probe can be designed to specifically hybridize to a nucleic acid comprising a polymorphic variant conferring a susceptibility to a neuropsychiatric disorder, e.g., increased severity of negative symptoms of schizophrenia or treatment response to an agonist of the glycine site of an NMDA receptor.

In some embodiments, restriction digest analysis can be used to assay for the existence of a polymorphic variant of a polymorphism, if alternate polymorphic variants of the polymorphism result in the creation or elimination of a restriction site. A sample containing genomic DNA is obtained from the individual. Polymerase chain reaction (PCR) can be used to amplify a region comprising the polymorphic site, and restriction fragment length polymorphism analysis is conducted (see Ausubel et al., Current Protocols in Molecular Biology, supra). The digestion pattern of the relevant DNA fragment indicates the presence or absence of a particular polymorphic variant of the polymorphism and is therefore indicative of susceptibility to a neuropsychiatric disorder, e.g., an increase or decrease in severity of negative symptoms of schizophrenia or treatment response to an agonist of the glycine site of an NMDA receptor.

Sequence analysis can also be used to detect specific polymorphic variants. A sample comprising DNA or RNA is obtained from the subject. PCR or other appropriate methods can be used to amplify a portion encompassing the polymorphic site, if desired. The sequence is then ascertained, using any standard method, and the presence of a polymorphic variant is determined.

Allele-specific oligonucleotides can also be used to assay for the presence of a polymorphic variant, e.g., through the use of dot-blot hybridization of amplified oligonucleotides with allele-specific oligonucleotide (ASO) probes (see, for example, Saiki et al., Nature (London) 324:163-166 (1986)). An “allele-specific oligonucleotide” (also referred to herein as an “allele-specific oligonucleotide probe”) is typically an oligonucleotide of approximately 10-50 base pairs, preferably approximately 15-30 base pairs, that specifically hybridizes to a nucleic acid region that contains a polymorphism. An allele-specific oligonucleotide probe that is specific for a particular polymorphism can be prepared using standard methods (see Ausubel et al., Current Protocols in Molecular Biology, supra).

Generally, to determine which of multiple polymorphic variants is present in a subject, a sample comprising DNA is obtained from the individual. PCR can be used to amplify a portion encompassing the polymorphic site. DNA containing the amplified portion may be dot-blotted, using standard methods (see Ausubel et al., Current Protocols in Molecular Biology, supra), and the blot contacted with the oligonucleotide probe. The presence of specific hybridization of the probe to the DNA is then detected. Specific hybridization of an allele-specific oligonucleotide probe (specific for a polymorphic variant indicative of increased severity of negative symptoms of schizophrenia or treatment response to an agonist of the glycine site of an NMDA receptor) to DNA from the subject is indicative of increased severity of negative symptoms of schizophrenia or treatment response to an agonist of the glycine site of an NMDA receptor.

In some embodiments, fluorescence polarization template-directed dye-terminator incorporation (FP-TDI) is used to determine which of multiple polymorphic variants of a polymorphism is present in a subject (Chen et al., Genome Research 9(5):492-498 (1999)). Rather than involving use of allele-specific probes or primers, this method employs primers that terminate adjacent to a polymorphic site, so that extension of the primer by a single nucleotide results in incorporation of a nucleotide complementary to the polymorphic variant at the polymorphic site.

Real-time pyrophosphate DNA sequencing is yet another approach to detection of polymorphisms and polymorphic variants (Alderborn et al., (2000) Genome Research, 10(8):1249-1258). Additional methods include, for example, PCR amplification in combination with denaturing high performance liquid chromatography (dHPLC) (Underhill, P. A., et al., Genome Research, Vol. 7, No. 10, pp. 996-1005, 1997).

The methods can include determining the genotype of a subject with respect to both copies of the polymorphic site present in the genome. For example, the complete genotype may be characterized as −/−, as −/+, or as +/+, where a minus sign indicates the presence of the reference or wild type sequence at the polymorphic site, and the plus sign indicates the presence of a polymorphic variant other than the reference sequence. If multiple polymorphic variants exist at a site, this can be appropriately indicated by specifying which ones are present in the subject. Any of the detection means described herein can be used to determine the genotype of a subject with respect to one or both copies of the polymorphism present in the subject's genome.

In some embodiments, it is desirable to employ methods that can detect the presence of multiple polymorphisms (e.g., polymorphic variants at a plurality of polymorphic sites) in parallel or substantially simultaneously. Oligonucleotide arrays represent one suitable means for doing so. Other methods, including methods in which reactions (e.g., amplification, hybridization) are performed in individual vessels, e.g., within individual wells of a multi-well plate or other vessel may also be performed so as to detect the presence of multiple polymorphic variants (e.g., polymorphic variants at a plurality of polymorphic sites) in parallel or substantially simultaneously according to certain embodiments of the invention.

### Probes

Nucleic acid probes can be used to detect and/or quantify the presence of a particular target nucleic acid sequence within a sample of nucleic acid sequences, e.g., as hybridization probes, or to amplify a particular target sequence within a sample, e.g., as a primer. Probes have a complimentary nucleic acid sequence that selectively hybridizes to the target nucleic acid sequence. In order for a probe to hybridize to a target sequence, the hybridization probe must have sufficient identity with the target sequence, i.e., at least 70%, e.g., 80%, 90%, 95%, 98% or more identity to the target sequence. The probe sequence must also be sufficiently long so that the probe exhibits selectivity for the target sequence over non-target sequences. For example, the probe will be at least 20, e.g., 25, 30, 35, 50, 100, 200, 300, 400, 500, 600, 700, 800, 900 or more, nucleotides in length. In some embodiments, the probes are not more than 30, 50, 100, 200, 300, 500, 750, or 1000 nucleotides in length. Probes are typically about 20 to about 1×10⁶ nucleotides in length. Probes include primers, which generally refers to a single-stranded oligonucleotide probe that can act as a point of initiation of template-directed DNA synthesis using methods such as PCR (polymerase chain reaction), LCR (ligase chain reaction), etc., for amplification of a target sequence. In some embodiments, the probe is a test probe, e.g., a probe that can be used to detect polymorphisms in a region described herein, e.g., polymorphisms as described herein. In some embodiments, the probe can bind to another marker sequence associated with schizophrenia, as described herein.

Control probes can also be used. For example, a probe that binds a less variable sequence, e.g., repetitive DNA associated with a centromere of a chromosome, can be used as a control. Probes that hybridize with various centromeric DNA and locus-specific DNA are available commercially, for example, from Vysis, Inc. (Downers Grove, Ill.), Molecular Probes, Inc. (Eugene, Oreg.), or from Cytocell (Oxfordshire, UK). Probe sets are available commercially, e.g., from Applied Biosystems, e.g., the Assays-on-Demand SNP kits. Alternatively, probes can be synthesized, e.g., chemically or in vitro, or made from chromosomal or genomic DNA through standard techniques. For example, sources of DNA that can be used include genomic DNA, cloned DNA sequences, somatic cell hybrids that contain one, or a part of one, human chromosome along with the normal chromosome complement of the host, and chromosomes purified by flow cytometry or microdissection. The region of interest can be isolated through cloning, or by site-specific amplification via the polymerase chain reaction (PCR). See, e.g., Nath and Johnson, Biotechnic. Histochem., 1998, 73(1):6-22, Wheeless et al., Cytometry 1994, 17:319-326, and U.S. Pat. No. 5,491,224.

In some embodiments, the probes are labeled, e.g., by direct labeling, with a fluorophore, an organic molecule that fluoresces after absorbing light of lower wavelength/higher energy. A directly labeled fluorophore allows the probe to be visualized without a secondary detection molecule. After covalently attaching a fluorophore to a nucleotide, the nucleotide can be directly incorporated into the probe with standard techniques such as nick translation, random priming, and PCR labeling. Alternatively, deoxycytidine nucleotides within the probe can be transaminated with a linker. The fluorophore then is covalently attached to the transaminated deoxycytidine nucleotides. See, e.g., U.S. Pat. No. 5,491,224.

Fluorophores of different colors can be chosen such that each probe in a set can be distinctly visualized. For example, a combination of the following fluorophores can be used: 7-amino-4-methylcoumarin-3-acetic acid (AMCA), TEXAS RED™ (Molecular Probes, Inc., Eugene, Oreg.), 5-(and-6)-carboxy-X-rhodamine, lissamine rhodamine B, 5-(and-6)-carboxyfluorescein, fluorescein-5-isothiocyanate (FITC), 7-diethylaminocoumarin-3-carboxylic acid, tetramethylrhodamine-5-(and-6)-isothiocyanate, 5-(and-6)-carboxytetramethylrhodamine, 7-hydroxycoumarin-3-carboxylic acid, 6-[fluorescein 5-(and-6)-carboxamido]hexanoic acid, N-(4,4-difluoro-5,7-dimethyl-4-bora-3a,4a diaza-3-indacenepropionic acid, eosin-5-isothiocyanate, erythrosin-5-isothiocyanate, and CASCADE™ blue acetylazide (Molecular Probes, Inc., Eugene, Oreg.). Fluorescently labeled probes can be viewed with a fluorescence microscope and an appropriate filter for each fluorophore, or by using dual or triple band-pass filter sets to observe multiple fluorophores. See, for example, U.S. Pat. No. 5,776,688. Alternatively, techniques such as flow cytometry can be used to examine the hybridization pattern of the probes. Fluorescence-based arrays are also known in the art.

In other embodiments, the probes can be indirectly labeled with, e.g., biotin or digoxygenin, or labeled with radioactive isotopes such as ³²P and ³H. For example, a probe indirectly labeled with biotin can be detected by avidin conjugated to a detectable marker. For example, avidin can be conjugated to an enzymatic marker such as alkaline phosphatase or horseradish peroxidase. Enzymatic markers can be detected in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase.

Oligonucleotide probes that exhibit differential or selective binding to polymorphic sites may readily be designed by one of ordinary skill in the art. For example, an oligonucleotide that is perfectly complementary to a sequence that encompasses a polymorphic site (i.e., a sequence that includes the polymorphic site, within it or at one end) will generally hybridize preferentially to a nucleic acid comprising that sequence, as opposed to a nucleic acid comprising an alternate polymorphic variant.

### Arrays and Uses Thereof

Arrays that include a substrate having a plurality of addressable areas and methods of using them are also provided. At least one area of the plurality includes a nucleic acid probe that binds specifically to a sequence comprising a polymorphism listed in Table 1, and can be used to detect the absence or presence of said polymorphism, e.g., one or more SNPs, microsatellites, minisatellites, or indels, as described herein, to determine a haplotype. For example, the array can include one or more nucleic acid probes that can be used to detect a polymorphism listed in Table 1. In some embodiments, the array further includes at least one area that includes a nucleic acid probe that can be used to specifically detect another marker associated with schizophrenia, as described herein. The substrate can be, e.g., a two-dimensional substrate known in the art such as a glass slide, a wafer (e.g., silica or plastic), a mass spectroscopy plate, or a three-dimensional substrate such as a gel pad. In some embodiments, the probes are nucleic acid capture probes.

Methods for generating arrays are known in the art and include, e.g., photolithographic methods (see, e.g., U.S. Pat. Nos. 5,143,854; 5,510,270; and 5,527,681), mechanical methods (e.g., directed-flow methods as described in U.S. Pat. No. 5,384,261), pin-based methods (e.g., as described in U.S. Pat. No. 5,288,514), and bead-based techniques (e.g., as described in PCT US/93/04145). The array typically includes oligonucleotide probes capable of specifically hybridizing to different polymorphic variants. According to the method, a nucleic acid of interest, e.g., a nucleic acid encompassing a polymorphic site, (which is typically amplified) is hybridized with the array and scanned. Hybridization and scanning are generally carried out according to standard methods. See, e.g., WO 92/10092 and WO 95/11995, and U.S. Pat. No. 5,424,186. After hybridization and washing, the array is scanned to determine the position on the array to which the nucleic acid hybridizes. The hybridization data obtained from the scan is typically in the form of fluorescence intensities as a function of location on the array.

Arrays can include multiple detection blocks (i.e., multiple groups of probes designed for detection of particular polymorphisms). Such arrays can be used to analyze multiple different polymorphisms. Detection blocks may be grouped within a single array or in multiple, separate arrays so that varying conditions (e.g., conditions optimized for particular polymorphisms) may be used during the hybridization. For example, it may be desirable to provide for the detection of those polymorphisms that fall within G-C rich stretches of a genomic sequence, separately from those falling in A-T rich segments. Additional description of use of oligonucleotide arrays for detection of polymorphisms can be found, for example, in U.S. Pat. Nos. 5,858,659 and 5,837,832. In addition to oligonucleotide arrays, cDNA arrays may be used similarly in certain embodiments of the invention.

The methods described herein can include providing an array as described herein;

contacting the array with a sample, e.g., a portion of genomic DNA that includes at least one marker described herein or another chromosome, e.g., including another region or marker associated with a neuropsychiatric disorder, e.g., schizophrenia, and detecting binding of a nucleic acid from the sample to the array. Optionally, the method includes amplifying nucleic acid from the sample, e.g., genomic DNA that includes a portion of a human chromosome described herein, and, optionally, a region that includes another region associated with schizophrenia, prior to or during contact with the array.

In some aspects, the methods described herein can include using an array that can ascertain differential expression patterns or copy numbers of one or more genes in samples from normal and affected individuals (see, e.g., Redon et al., Nature 444(7118):444-54 (2006)). For example, arrays of probes to a marker described herein can be used to measure polymorphisms between DNA from a subject having a neuropsychiatric disorder, e.g., schizophrenia, and control DNA, e.g., DNA obtained from an individual who does not have schizophrenia, and has no risk factors for schizophrenia. Since the clones on the array contain sequence tags, their positions on the array are accurately known relative to the genomic sequence. Different hybridization patterns between DNA from an individual afflicted with schizophrenia and DNA from a normal individual at areas in the array corresponding to markers as described herein, and, optionally, one or more other regions associated with schizophrenia, are indicative of increased severity of negative symptoms of schizophrenia or treatment response to an agonist of the glycine site of an NMDA receptor. Methods for array production, hybridization, and analysis are described, e.g., in Snijders et al., (2001) Nat. Genetics 29:263-264; Klein et al., (1999) Proc. Natl Acad. Sci. U.S.A. 96:4494-4499; Albertson et al., (2003) Breast Cancer Research and Treatment 78:289-298; and Snijders et al. “BAC microarray based comparative genomic hybridization.” In: Zhao et al. (Eds.), Bacterial Artificial Chromosomes: Methods and Protocols, Methods in Molecular Biology, Humana Press, 2002. Real time quantitative PCR can also be used to determine copy number.

In another aspect, the invention features methods of determining the absence or presence of an allele or a haplotype associated with a neuropsychiatric disorder, e.g., a negative symptom of schizophrenia, as described herein, using an array described above.

The methods include providing a two dimensional array having a plurality of addresses, each address of the plurality being positionally distinguishable from each other address of the plurality having a unique nucleic acid capture probe, contacting the array with a first sample from a test subject who is suspected of having or being at risk for a neuropsychiatric disorder, e.g., schizophrenia, and comparing the binding of the first sample with one or more references, e.g., binding of a sample from a subject who is known to have the neuropsychiatric disorder, e.g., schizophrenia, and/or binding of a sample from a subject who is unaffected, e.g., a control sample from a subject who neither has, nor has any risk factors for the neuropsychiatric disorder, e.g., schizophrenia. In some embodiments, the methods include contacting the array with a second sample from a subject who has the neuropsychiatric disorder, e.g., schizophrenia; and comparing the binding of the first sample with the binding of the second sample. In some embodiments, the methods include contacting the array with a third sample from a cell or subject that does not have schizophrenia and is not at risk for schizophrenia; and comparing the binding of the first sample with the binding of the third sample. In some embodiments, the second and third samples are from first or second-degree relatives of the test subject. Binding, e.g., in the case of a nucleic acid hybridization, with a capture probe at an address of the plurality, can be detected by any method known in the art, e.g., by detection of a signal generated from a label attached to the nucleic acid.

### Kits

Also within the scope of the invention are kits comprising a probe that hybridizes with a region of human chromosome as described herein and can be used to detect a polymorphism described herein. The kit can include one or more other elements including: instructions for use; and other reagents, e.g., a label, or an agent useful for attaching a label to the probe. Instructions for use can include instructions for diagnostic applications of the probe for predicting response to treatment of a neuropsychiatric disorder, e.g., a negative symptom of schizophrenia, in a method described herein. Other instructions can include instructions for attaching a label to the probe, instructions for performing in situ analysis with the probe, and/or instructions for obtaining a sample to be analyzed from a subject. As discussed above, the kit can include a label, e.g., any of the labels described herein. In some embodiments, the kit includes a labeled probe that hybridizes to a region of human chromosome as described herein, e.g., a labeled probe as described herein.

The kit can also include one or more additional probes that hybridize to the same chromosome or another chromosome or portion thereof that can have an abnormality associated with severity of negative symptoms of schizophrenia. A kit that includes additional probes can further include labels, e.g., one or more of the same or different labels for the probes. In other embodiments, the additional probe or probes provided with the kit can be a labeled probe or probes. When the kit further includes one or more additional probe or probes, the kit can further provide instructions for the use of the additional probe or probes.

Kits for use in self-testing can also be provided. For example, such test kits can include devices and instructions that a subject can use to obtain a sample, e.g., of buccal cells or blood, without the aid of a health care provider. For example, buccal cells can be obtained using a buccal swab or brush, or using mouthwash.

Kits as provided herein can also include a mailer, e.g., a postage paid envelope or mailing pack, that can be used to return the sample for analysis, e.g., to a laboratory. The kit can include one or more containers for the sample, or the sample can be in a standard blood collection vial. The kit can also include one or more of an informed consent form, a test requisition form, and instructions on how to use the kit in a method described herein. Methods for using such kits are also included herein. One or more of the forms, e.g., the test requisition form, and the container holding the sample, can be coded, e.g., with a bar code, for identifying the subject who provided the sample.

In some embodiments, the kits can include one or more reagents for processing a biological sample. For example, a kit can include reagents for isolating mRNA or genomic DNA from a biological sample and/or reagents for amplifying isolated mRNA (e.g., reverse transcriptase, primers for reverse transcription or PCR amplification, or dNTPs) and/or genomic DNA. The kits can also, optionally, contain one or more reagents for detectably-labeling an mRNA, mRNA amplicon, genomic DNA or DNA amplicon, which reagents can include, e.g., an enzyme such as a Klenow fragment of DNA polymerase, T4 polynucleotide kinase, one or more detectably-labeled dNTPs, or detectably-labeled gamma phosphate ATP (e.g., ³³P-ATP).

In some embodiments, the kits can include a software package for analyzing the results of, e.g., a microarray analysis or expression profile.

### Databases

Also provided herein are databases that include a list of polymorphisms as described herein, and wherein the list is largely or entirely limited to polymorphisms identified as useful in performing genetic diagnosis of or determination of severity of a neuropsychiatric disorder, e.g., severity of negative symptoms of schizophrenia. The list is stored, e.g., on a flat file or computer-readable medium. The databases can further include information regarding one or more subjects, e.g., whether a subject is affected or unaffected, clinical information such as age of onset of symptoms, any treatments administered and outcomes (e.g., data relevant to pharmacogenomics, diagnostics, or theranostics), and other details, e.g., about the disorder in the subject, or environmental or other genetic factors. The databases can be used to detect correlations between a particular haplotype and the information regarding the subject, e.g., to detect correlations between a haplotype and a particular phenotype, or treatment response.

### Engineered Cells

Also provided herein are engineered cells that harbor one or more polymorphism described herein, e.g., two, three, or four polymorphisms that constitute a haplotype associated with severity of a neuropsychiatric disorder, e.g., severity of negative symptoms of schizophrenia, or treatment response to an agonist of the glycine site of an NMDA receptor. Such cells are useful for studying the effect of one or more polymorphism on physiological function, and for identifying and/or evaluating potential therapeutic agents for the treatment of a neuropsychiatric disorder, e.g., a negative symptom of schizophrenia, e.g., glycine, D-alanine, D-serine, D-cycloserine, N-methylglycine, and RG1678.

As one example, included herein are cells in which one of the various alleles of the genes described herein has been re-created that are associated with a neuropsychiatric disorder, e.g., a negative symptom of schizophrenia. Methods are known in the art for generating cells, e.g., by homologous recombination between the endogenous gene and an exogenous DNA molecule introduced into a cell, e.g., a cell of an animal. In some embodiments, the cells can be used to generate transgenic animals using methods known in the art.

The cells are preferably mammalian cells, e.g., epithelial or endothelial type cells, in which an endogenous gene has been altered to include a polymorphism as described herein. Techniques such as targeted homologous recombinations can be used to insert the heterologous DNA as described in, e.g., Chappel, U.S. Pat. No. 5,272,071; WO 91/06667, published in May 16, 1991.

### Subjects to be Treated

A subject can be selected on the basis that they have, or are at risk of developing, a neuropsychiatric disorder, e.g., schizophrenia. It is well within the skills of an ordinary practitioner to recognize a subject that has, or is at risk of developing, a neuropsychiatric disorder, e.g., schizophrenia. A subject that has, or is at risk of developing, schizophrenia is one having one or more symptoms of the condition or one or more risk factors for developing the condition. Symptoms of schizophrenia are known to those of skill in the art and include, without limitation, loss of interest in everyday activities, appearing to lack emotion, reduced ability to plan or carry out activities, neglect of personal hygiene, social withdrawal, loss of motivation, delusions, hallucinations, thought disorder, problems with making sense of information, difficulty paying attention, memory problems, disorganized behavior, depression, and mood swings. A subject that has, or is at risk of developing, schizophrenia is one with known risk factors such as complications during pregnancy or birth (e.g., a child who experiences oxygen deprivation during pregnancy, bleeding during pregnancy, maternal malnutrition, infections during pregnancy, long labor, prematurity, and low birth weight), stress, poor nutrition, and certain family backgrounds.

The methods are effective for a variety of subjects including mammals, e.g., humans and other animals, such as laboratory animals, e.g., mice, rats, rabbits, or monkeys, or domesticated and farm animals, e.g., cats, dogs, goats, sheep, pigs, cows, or horses.

### NMDA Agonists

The treatment method of the invention entails administering to a subject diagnosed as having a neuropsychiatric disorder a pharmaceutical composition comprising a therapeutically effective amount of an agonist of the glycine site of the NMDA receptor, which agonist is relatively selective for the glycine site of the NMDA receptor, or a glycine uptake inhibitor, compared with an inhibitory glycine receptor or any other receptor. For example, suitable pharmaceutical compositions may include (i) glycine and/or (ii) D-alanine and/or (iii) D-serine and/or (iv) N-methylglycine.

Glycine, D-alanine, and D-serine are commercially available (e.g., from Sigma-Aldrich Co., St. Louis, MO). Such compositions typically contain from about 0.1 to 90% by weight (such as 1 to 20% or 1 to 10%) of glycine, D-alanine, D-serine, or N-methylglycine in a pharmaceutically acceptable carrier. Regardless of the concentration of glycine, D-alanine, or D-serine in the pharmaceutical composition, glycine and/or D-alanine and/or D-serine and/or N-methylglycine is administered to the subject at a dosage of 10 mg to 100 g. More typically, glycine and/or D-alanine and/or D-serine and/or N-methylglycine is administered at a dosage of 100 mg to 10 g. Generally, treatment continues for at least several weeks to several years or life-long as needed.

In an alternative method for treating a neuropsychiatric disorder in a subject, a pharmaceutical composition comprising D-cycloserine in an amount equivalent to a dosage of 10 to 500 mg is administered once a week to a subject in need of such treatment. For example, the dosage can be in an amount of 20 to 200 mg, such as 30 to 100 mg (e.g., 40 mg, 50 mg, 60 mg, or 70 mg). D-cycloserine is commercially available from Sigma-Aldrich Co. (St. Louis, Mo.). Generally, treatment continues for at least one week and can continue for several years or life-long as needed to control the subject's symptoms.

In all of the methods of the invention, glycine, D-alanine, D-serine, and/or D-cycloserine and/or N-methylglycine can be substituted with a modified version of the amino acid, such as a salt, ester, alkylated form, or a precursor of the amino acid. For example, the amino acid can be in the form of a sodium salt, potassium salt, calcium salt, magnesium salt, zinc salt, or ammonium salt. Such salt forms of glycine, D-alanine, D-serine, D-cycloserine, and N-methylglycine can be made in accordance with conventional methods (see, e.g., Organic Chemistry, pgs. 822-823, Morrison and Boyd, ed., Fifth Edition, Allyn and Bacon, Inc., Newton, Mass.). Other modified forms of glycine, D-alanine, D-serine, D-cycloserine, and N-methylglycine also can be used in the methods of the invention. For example, the carboxyl group of the amino acid can be converted to an ester group by reaction with an alcohol in accordance with standard esterification methods (Id. at 841-843). For example, alcohols having 1-20 carbon atoms can be used to produce an ester of glycine, D-alanine, D-serine, D-cycloserine, or N-methylglycine for use in the present methods (e.g., methyl-, ethyl-, propyl-, isopropyl-, butyl-, isobutyl-, sec-butyl-, tert-butyl-, pentyl-, isopentyl-, tert-pentyl-, hexyl-, heptyl-, octyl-, decyl-, dodecyl-, tetradecyl-, hexadecyl-, octadecyl-, and phenyl-alcohols can be used). In another variation, the amino group of the amino acid can be alkylated, using conventional methods, to produce a secondary or tertiary amino group by ammonolysis of halides or reductive amination (Id. at 939-948). For example, an alkyl group having 1-20 carbon atoms can be added to the amino acid to produce an alkylated amino acid (e.g., methyl-, ethyl-, propyl-, isopropyl-, butyl-, isobutyl-, sec-butyl-, tert-butyl-, pentyl-, isopentyl-, tert-pentyl-, hexyl-, heptyl-, octyl-, decyl-, dodecyl-, tetradecyl-, hexadecyl-, octadecyl- and phenyl-groups can be added to the amino acid). D-phosphoserine and L-phosphoserine are examples of precursors of D-serine, and are commercially available (e.g., from Sigma-Aldrich, St. Louis, Mo.). N,N,N-trimethylglycine (betaine) and N,N-dimethylglycine are examples of precursors of N-methylglycine.

In all of the methods described herein, appropriate dosages of NMDA agonists, e.g., glycine, D-alanine, D-serine, D-cycloserine, and N-methylglycine, can readily be determined by those of ordinary skill in the art of medicine by monitoring the patient for signs of disease amelioration or inhibition, and increasing or decreasing the dosage and/or frequency of treatment as desired.

The pharmaceutical compositions can be administered to the patient by any, or a combination, of several routes, such as oral, intravenous, trans-mucosal (e.g., nasal, vaginal, etc.), pulmonary, transdermal, ocular, buccal, sublingual, intraperitoneal, intrathecal, intramuscular, parenteral, or long term depot preparation. Solid compositions for oral administration can contain suitable carriers or excipients, such as corn starch, gelatin, lactose, acacia, sucrose, microcrystalline cellulose, kaolin, mannitol, dicalcium phosphate, calcium carbonate, sodium chloride, lipids, alginic acid, or ingredients for controlled slow release. Disintegrators that can be used include, without limitation, micro-crystalline cellulose, corn starch, sodium starch glycolate and alginic acid. Tablet binders that may be used include, without limitation, acacia, methylcellulose, sodium carboxymethylcellulose, polyvinylpyrrolidone (Povidone), hydroxypropyl methylcellulose, sucrose, starch, and ethylcellulose.

Liquid compositions for oral administration prepared in water or other aqueous vehicles can include solutions, emulsions, syrups, and elixirs containing, together with the active compound(s), wetting agents, sweeteners, coloring agents, and flavoring agents. Various liquid and powder compositions can be prepared by conventional methods for inhalation into the lungs of the patient to be treated.

Injectable compositions may contain various carriers such as vegetable oils, dimethylacetamide, dimethylformamide, ethyl lactate, ethyl carbonate, isopropyl myristate, ethanol, and polyols (glycerol, propylene glycol, liquid polyethylene glycol, and the like). For intravenous injections, the compounds may be administered by the drip method, whereby a pharmaceutical composition containing the active compound(s) and a physiologically acceptable excipient is infused. Physiologically acceptable excipients may include, for example, 5% dextrose, 0.9% saline, Ringer's solution or other suitable excipients. For intramuscular preparations, a sterile composition of a suitable soluble salt form of the compound can be dissolved and administered in a pharmaceutical excipient such as Water-for-Injection, 0.9% saline, or 5% glucose solution, or depot forms of the compounds (e.g., decanoate, palmitate, undecylenic, enanthate) can be dissolved in sesame oil. Alternatively, the pharmaceutical composition can be formulated as a chewing gum, lollipop, or the like.

The subjects can also be those undergoing any of a variety of neuropsychiatric treatments. Thus, for example, subjects can be those being treated with one or more antipsychotic agents (e.g., haloperidol, chlorpromazine, triflupromazine, chlorprothixene, thiothixene, clozapine, risperidone, and aripiprazole), NMDA receptor agonists (e.g., glycine, D-alanine, D-serine), NMDA receptor partial agonist (e.g., D-cycloserine), glycine reuptake inhibitors (e.g., N-methylglycine and RG1678), selective serotonin reuptake inhibitors (e.g., fluoxetine, citalopram, dapoxetine, alopram, sertraline, and paroxetine), other glutamatergic compounds, estrogen, clozapine, acetylcholinesterase inhibitors (e.g., galantamine, rivastigmine, and donepezil), folate, and vitamin B12. The invention is further described in the following examples, which do not limit the scope of the invention described in the claims.

### EXAMPLES

D-amino acid oxidase activator (DAOA, also known as G72) is a protein enriched in various parts of brain, spinal cord, and testis. G72 increases activity of D-amino acid oxidase (DAO), an enzyme that metabolizes D-serine, the primary endogenous agonist at the glycine site of the NMDA receptor in brain. Meta-analyses have established G72 as a risk gene for schizophrenia (Detera-Wadleigh and McMahon, Biol Psychiatry 60:106-14, 2006.). In the presence of DAOA, D-serine metabolism is increased (Chumakov et al., Proc. Natl. Acad. Sci. USA 99:13675-13680, 2002). Elevated expression of G72 mRNA has been found in schizophrenia brain (Korostishevsky et al., Biol Psychiatry 56: 169-76, 2004), as well as increased activity of D-amino acid oxidase (DAO) and decreased levels of D-serine in CSF and serum of patients with schizophrenia (Hashimoto et al., Prog Neuropsychopharmacol Biol Psychiatry 29:767-9, 2005; Hashimoto et al., Arch Gen Psychiatry 60:572-6, 2003). Hence, G72 genotype is a marker for endogenous activity at the glycine site of the NMDA receptor and linkage of this gene with schizophrenia supports the role of NMDA receptor hypofunction as an etiologic mechanism.

GCPII, also called FOLH1, is a glutamate carboxypeptidase that regulates glutamatergic NMDA receptor activity in the brain. The 484T>C variant is located in exon 2 of the structural transmembrane region and confers a 75Tyr>His amino acid change. Here, the 484C variant was associated with more severe negative symptoms.

Of note, GCPII is expressed in the brain where it is known as NAALADase and cleaves n-acetylaspartylglutamate (NAAG) into n-acetylaspartate (NAA) and glutamate (Bacich et al., Mamm Genome 12:117-123, 2001). NAA is a marker of neuronal integrity for which hippocampal and prefrontal levels are consistently reduced in magnetic resonance spectroscopy studies of schizophrenia (Marenco et al., Adv Exp Med Biol 576:227-40, 2006), while glutamatergic dysfunction in schizophrenia is well established (Coyle JT, Cell Mol Neurobiol 26:365-384, 2006). GCPII therefore could represent an important target in schizophrenia pathophysiology.

Although common genetic variants may contribute approximately one-third of the total genetic liability in schizophrenia (Purcell et al., Nature 460:748-752, 2009), effects of individual variants are small, and many variants that show consistent replication in candidate gene studies are still not strong enough to reach genome-wide significance. Understanding how variants of small effect combine to exert clinically meaningful influences on schizophrenia phenotypes will be critical in deciphering the genetic architecture of the disorder. Increasingly, genome wide association studies and other high-throughput genetic investigations are relying on metabolic pathway analyses in order to pool risk variants into biologically meaningful contexts (Mill et al., Am J Hum Genet 82:696-711, 2008; O′Dushlaine et al., in press). Described herein are genetic variants involved in NMDA receptor activity and their contribution to negative symptom risk in schizophrenia. Subjects who possess a greater number of functional genetic variants are particularly susceptible for negative symptoms, perhaps reflecting a cumulative effect of these variants on downstream reactions. The approach of canvassing genetic variants in implicated biological pathways to generate cumulative risk scores holds promise in resolving the so-called “missing heritability” in schizophrenia and other complex genetic disorders in psychiatry (Maher, Nature 456:18-21, 2008) just as in the present study, where the net effects of related variants outweigh the influence of a single variant on negative symptom severity.

Even among subjects who carry multiple risk alleles, negative symptoms can be ameliorated in the presence of agonists of the glycine site of the NMDA receptor.

### Schizophrenia

Schizophrenia is a chronic, severe, and disabling brain disease. Approximately 1 to 1.5 percent of the population develops schizophrenia during their lifetime; more than 2 million Americans suffer from the illness in a given year. Although schizophrenia affects men and women with equal frequency, the disorder often appears earlier in men, usually in the late teens or early twenties, than in women, who are generally affected in the twenties to early thirties. People with schizophrenia often suffer terrifying symptoms such as hearing internal voices not heard by others, or believing that other people are reading their minds, controlling their thoughts, or plotting to harm them. These symptoms may leave them fearful and withdrawn. Their speech and behavior can be so disorganized that they may be incomprehensible or frightening to others. Available treatments can relieve many symptoms, but most people with schizophrenia continue to suffer some symptoms throughout their lives; it has been estimated that no more than one in five individuals recovers completely.

Negative symptoms of schizophrenia, which include apathy, impoverished speech, flattened affect, and social withdrawal, contribute greatly to functional disability in schizophrenia and are not substantially improved by antipsychotic medications (Goff et al., Schizophrenia. Med Clin North Am 85:663-689, 2001; Lieberman et al., N Engl J Med 353:1209-1223, 2005; Mohamed et al., Am J Psychiatry 165:978-987, 2008).

The first signs of schizophrenia often appear as confusing, or even shocking, changes in behavior. Coping with the symptoms of schizophrenia can be especially difficult for family members who remember how involved or vivacious a person was before they became ill. The sudden onset of severe psychotic symptoms is referred to as an acute phase of schizophrenia. Psychosis, a common condition in schizophrenia, is a state of mental impairment marked by hallucinations, which are disturbances of sensory perception, and/or delusions, which are false yet strongly held personal beliefs that result from an inability to separate real from unreal experiences. Less obvious symptoms, such as social isolation or withdrawal, or unusual speech, thinking, or behavior, may precede, be seen along with, or follow the psychotic symptoms.

Some people have only one such psychotic episode; others have many episodes during a lifetime, but lead relatively normal lives during the interim periods. However, an individual with chronic schizophrenia, or a continuous or recurring pattern of illness, often does not fully recover normal functioning and typically requires long-term treatment, generally including medication, to control the symptoms.

Schizophrenia is found all over the world. The severity of the symptoms and long-lasting, chronic pattern of schizophrenia often cause a high degree of disability. Medications and other treatments for schizophrenia, when used regularly and as prescribed, can help reduce and control the distressing symptoms of the illness. However, some people are not greatly helped by available treatments or may prematurely discontinue treatment because of unpleasant side effects or other reasons. Even when treatment is effective, persisting consequences of the illness, lost opportunities, stigma, residual symptoms, and medication side effects may be very troubling.

Study procedures were approved by the Partners HealthCare and Massachusetts Department of Mental Health institutional review boards, and all participants provided written informed consent. A diagnosis of schizophrenia was confirmed by a consensus diagnostic conference based on results from a clinical diagnostic interview, chart review, and review of clinical history with treating physicians.

Subjects were administered the Positive and Negative Syndrome Scale (PANSS) (Kay et al., Schizophrenia Bulletin 13:261-276) to assess symptom severity by trained raters who were blind to genotype.

DNA was obtained from blood samples and genotyped for variants across two genes: G72 and GCPII. Specific variants were selected on the basis of (1) common occurrence in the general population (minor allele frequency >0.2), (2) coding for non-synonymous mutations in amino acid sequences, and (3) previous support in the literature for an association with schizophrenia. No additional genetic variants were studied. Genotyping was conducted using the MASSARRAY® platform (Sequenom, San Diego, Calif.) using the nucleotide primers shown in Table 2.

TABLE 2
Nucleotide Primers to Detect SNPs

| SNP | Forward Primer | Reverse Primer | Extension Primer |
|---|---|---|---|
| rs3916971 | ACGTTGGATGTTGGC | ACGTTGGATGTCCTC | AACTAATCCTTGCTCAC |
| | AACTAATCCTTGCTC | TTCCCATGCTGTTTC | ATTT |
| | (SEQ ID NO: 3) | (SEQ ID NO: 4) | (SEQ ID NO: 5) |
| rs202676 | ACGTTGGATGCTTTG | ACGTTGGATGGTCCA | TAAAGCTGAGAACATCA |
| | AGGAAATCATGGAAG | TATAAACTTTCGAGG | AGAAGTTCTTA |
| | (SEQ ID NO: 6) | (SEQ ID NO: 7) | (SEQ ID NO: 8) |

Results

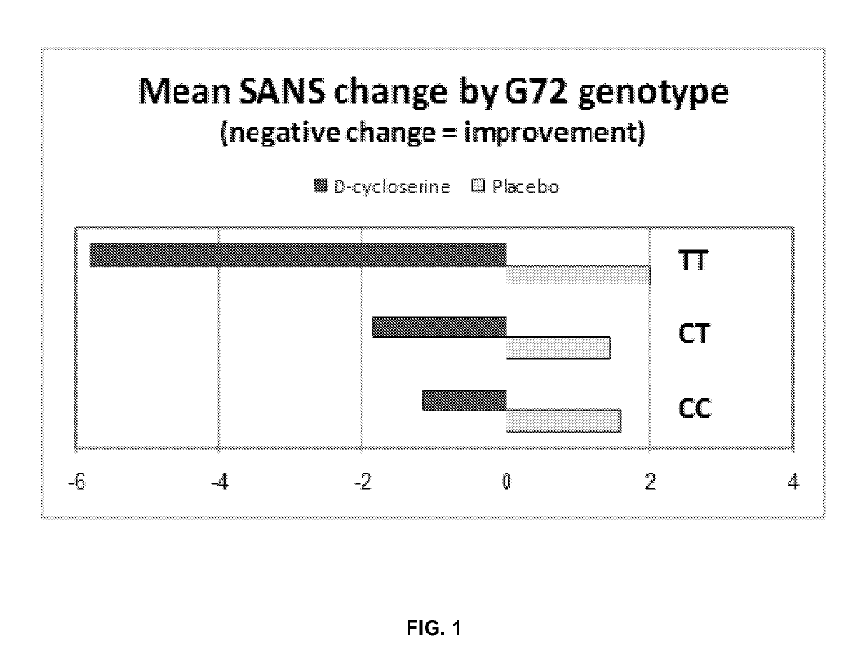

Because studies in animals have demonstrated tachyphylaxis with repeated daily dosing of D-cycloserine (Parnas et al., Neurobiol Learn Mem 83:224-31, 2005; Quartermain et al., Eur J Pharm 257:7-12, 1994), whereas a single dose of D-cycloserine produces persistent neuroplastic synaptic changes including recovery from brain injury in mice (Yaka et al., FASEB J 21:2033-41, 2007) and enhanced cortical motor neuroplasticity measured by rTMS in humans (Nitsche et al., Neuropsychopharmacology 29:1573-8, 2004), a placebo controlled add-on trial was conducted of once-weekly D-cycloserine 50 mg in 38 stable schizophrenia patients treated with first and second generation antipsychotics except clozapine (Goff et al., Schizophr Res 106:320-7, 2008). Negative symptoms were measured by the modified SANS total score at baseline and at week 8, seven days after the last dose of study drug. D-cycloserine significantly improved negative symptoms (effect size 0.7); 30% of patients exhibited a 20% or greater improvement in SANS total score compared to 11% in the placebo group. The sample was subsequently enlarged to 45 subjects and G72 genotype was examined as a predictor of response. G72 is the most-validated schizophrenia risk gene that influences activity at the glycine site of the NMDA receptor complex. One G72 SNP, rs3916971 (M21), that achieved significant association with schizophrenia risk in a meta-analysis performed by “SzGene” (Bertram, Schizophr Bull. 34:806-12, 2008) was examined. In the enlarged sample of 45 subjects, the effect size of D-cycloserine improvement of negative symptoms compared to placebo increased to 0.9 and G72 genotype produced a drug x genotype effect size of 0.7. Within subjects receiving D-cycloserine, G72 genotype predicted response with an effect size of 0.9 and within subjects with the TT genotype, the effect size of D-cycloserine improvement of negative symptoms vs. placebo was 2.3 (FIG. 1).

G72 genotype (rs3916971) predicted negative symptoms at a trend level for all subjects and significantly in Caucasians. For all genotyped subjects with negative symptom scores (n=352), there is a trend effect of T allele load on negative symptom scores (C/C=17.2, C/T=17.5, T/T=18.7; p=0.064 for linear regression). In Caucasian subjects (n=262), the effect is statistically significant (p=0.038).

GCPII genotype (rs202676) adds to the predictive power of the G72 genotype (rs3916971). GCPII were divided into two genotype groups as there are only a few C/C's (C carrier, n=100; T/T, n=162); T/T is protective. There were three G72 groups (C/C=92; C/T=129; T/T=41); C/C is protective. Interaction between the two genotypes is significant (p=0.01 via linear regression, and p=0.04 via ANOVA). The group that separates out (G72 C/C+GCPII T/T) is large (n=62). The interaction term for the entire sample is p=0.09 for linear regression, and p=0.06 for ANOVA.

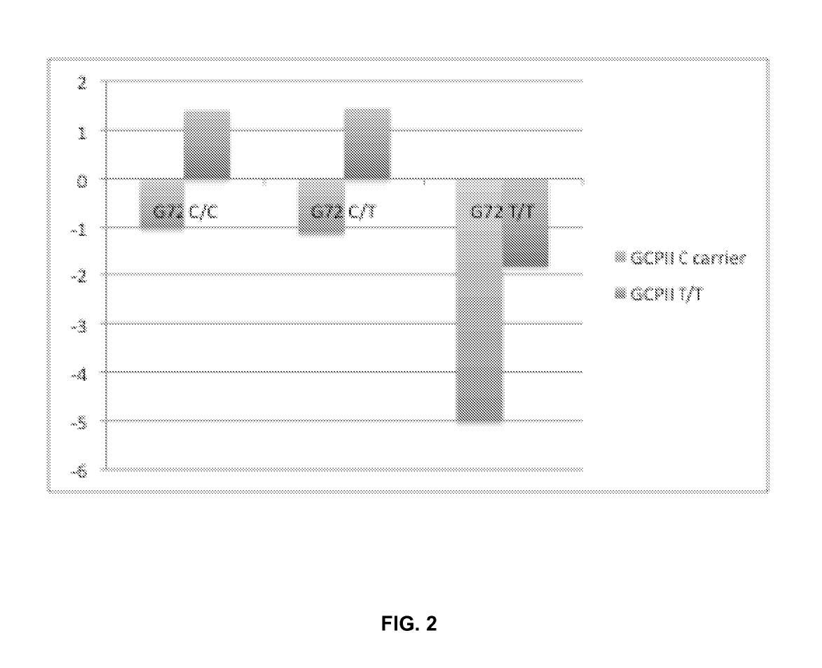

GCPII significantly interacts with G72 in predicting response to once-weekly 50 mg D-cycloserine treatment. These two genes also predict severity of negative symptoms as both modulate NMDA receptor activity. Negative scores indicate an improvement in SANS (FIG. 2).

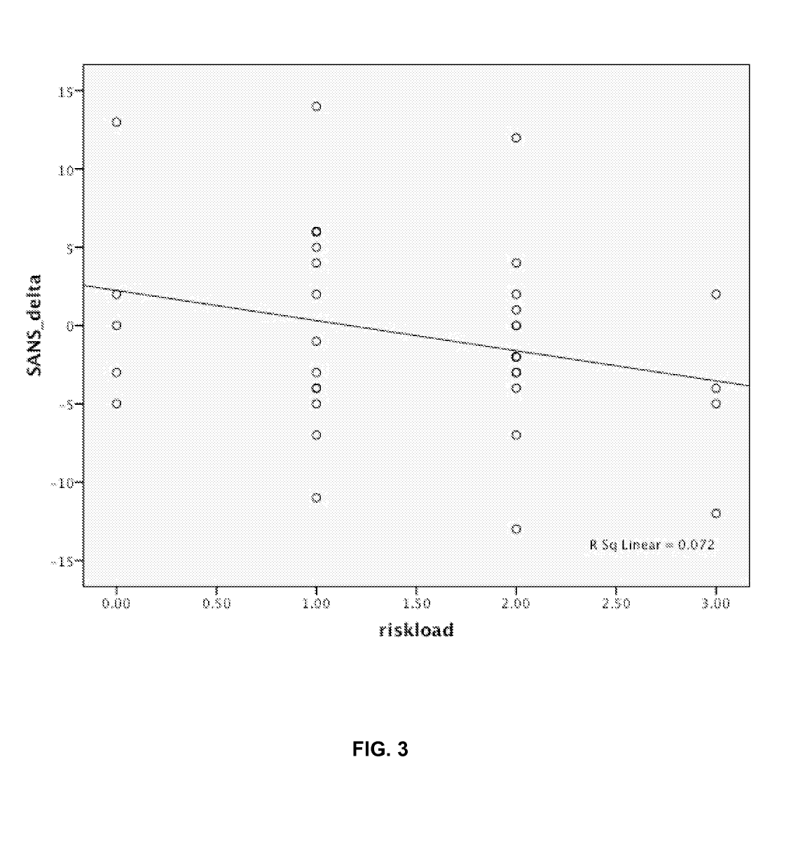

Based on prior negative symptom studies (see, e.g., U.S. Application No. 61/419,742), the protective genotype for GCPII is T/T and the risk genotype is “C.” For G72, C/C is protective and “T” carriers have the risk genotype. Subjects who have the risk genotypes are expected to show the most improvement. The relationship between change in SANS and number of risk alleles across the G72 and GCPII SNPs is shown in FIG. 3 (p=0.11).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating a subject diagnosed as having a neuropsychiatric disorder characterized by attenuated N-methyl-D-aspartate (NMDA) neurotransmission, the method comprising:
determining the presence of one or more alleles at rs3916971 and rs202676 in a sample comprising genomic DNA from the subject;
selecting a treatment for the subject based on the presence of the one or more alleles; and
treating the subject with the selected treatment.

2. The method of claim 1, wherein if a “T” at rs3916971 or a “C” at rs202676 is present, then a treatment comprising prescribing or administering an agonist of the glycine site of an NMDA receptor to the subject is selected.

3. The method of claim 1, wherein if a “T” at rs3916971 and a “C” at rs202676 are present, then a treatment comprising prescribing or administering an agonist of the glycine site of an NMDA receptor to the subject is selected.

4. The method of claim 1, wherein the method comprises detecting the presence of four alleles, wherein the four alleles consist of two alleles at each of rs3916971 and rs202676.

5. The method of claim 4, wherein if a “T” at rs3916971 and a “C” at rs202676 are present, and one or more additional alleles are a “T” at rs3916971 or a “C” at rs202676, then a treatment comprising prescribing or administering an agonist of the glycine site of an NMDA receptor to the subject is selected.

6. The method of claim 4, wherein if two alleles are a “T” at rs3916971 and two alleles are a “C” at rs202676, then a treatment comprising prescribing or administering an agonist of the glycine site of an NMDA receptor to the subject is selected.

7. The method of claim 1, wherein the subject is diagnosed as having a negative symptom of schizophrenia.

8. The method of claim 7, wherein the negative symptom is selected from the group consisting of apathy, impoverished speech, flattened affect, and social withdrawal.

9. The method of claim 1, wherein the subject is diagnosed as having a neuropsychiatric disorder selected from the group consisting of Alzheimer's disease, autism, depression, and attention deficit disorder.

10. The method of claim 1, wherein the selected treatment comprises prescribing or administering an agonist of the glycine site of an NMDA receptor to the subject.

11. The method of claim 10, wherein the agonist of the glycine site of an NMDA receptor is selected from the group consisting of glycine, a salt of glycine, an ester of glycine, alkylated glycine, a precursor of glycine, D-alanine, a salt of D-alanine, an ester of D-alanine, alkylated D-alanine, a precursor of D-alanine, D-serine, a salt of D-serine, an ester of D-serine, alkylated D-serine, and a precursor of D-serine.

12. The method of claim 10, wherein the agonist of the glycine site of an NMDA receptor is selected from the group consisting of D-cycloserine, a salt of D-cycloserine, an ester of D-cycloserine, alkylated D-cycloserine, and a precursor of D-cycloserine.

13. The method of claim 10, wherein the agonist of the glycine site of an NMDA receptor is selected from the group consisting of N-methylglycine, a salt of N-methylglycine, an ester of N-methylglycine, alkylated N-methylglycine, and a precursor of N-methylglycine.

14. The method of claim 10, wherein the selected treatment further comprises prescribing or administering an antipsychotic to the subject.

15. A method comprising:
assaying for the presence of one or more alleles at rs3916971 and rs202676 in a biological sample comprising genomic DNA from a subject diagnosed as having a negative symptom of schizophrenia; and
transmitting to a recipient a report on the presence of the one or more alleles.

16. The method of claim 15, wherein the method further comprises selecting a treatment for reducing the negative symptom in the subject based on the presence of the one or more alleles.

17. The method of claim 15, wherein if a “T” at rs3916971 and a “C” at rs202676 are present, then a treatment comprising prescribing or administering an agonist of the glycine site of an NMDA receptor to the subject is selected.

18. The method of claim 15, wherein the method comprises detecting the presence of four alleles, wherein the four alleles consist of two alleles at each of rs3916971 and rs202676.

19. The method of claim 15, wherein if a “T” at rs3916971 and a “C” at rs202676 are present, and one or more additional alleles are a “T” at rs3916971 or a “C” at rs202676, then a treatment comprising prescribing or administering an agonist of the glycine site of an NMDA receptor to the subject is selected.

20. The method of claim 15, wherein the selected treatment comprises prescribing or administering an agonist of the glycine site of an NMDA receptor to the subject.


SEQUENCE LISTING (8 sequences)

SEQ ID NO: 1 - DNA, 53 bp, Homo sapiens
acacctggcacatagtaatagatcatctaaatgtgagcaaggattagttgcca

SEQ ID NO: 2 - DNA, 53 bp, Homo sapiens
aagctgagaacatcaagaagttcttactagtaagtacatcctcgaaagtttat

SEQ ID NO: 3 - DNA, 30 bp, Artificial
  laboratory-synthesized DNA primers
acgttggatgttggcaactaatccttgctc

SEQ ID NO: 4 - DNA, 30 bp, Artificial
  laboratory-synthesized DNA primers
acgttggatgtcctcttcccatgctgtttc

SEQ ID NO: 5 - DNA, 21 bp, Artificial
  laboratory-synthesized DNA primers
aactaatccttgctcacattt

SEQ ID NO: 6 - DNA, 30 bp, Artificial
  laboratory-synthesized DNA primers
acgttggatgctttgaggaaatcatggaag

SEQ ID NO: 7 - DNA, 30 bp, Artificial
  laboratory-synthesized DNA primers
acgttggatggtccatataaactttcgagg

SEQ ID NO: 8 - DNA, 28 bp, Artificial
  laboratory-synthesized DNA primers
taaagctgagaacatcaagaagttctta